(19) United States
(12) Patent Application Publication
(10) Pub. No.: US 2023/0054346 A1
(43) Pub. Date: Feb. 23, 2023

(54) COMPOSITION COMPRISING HYALURONIC ACID AND A POLYOL AND/OR CARBOXYMETHYL CELLULOSE

(71) Applicant: IBSA PHARMA SAS, ANTIBES (FR)

(72) Inventors: Yannis GUILLEMIN, MONTAUROUX (FR); Dominique VACHER, Savosa (CH); Jean-Noel GOUZE, Vallauris (FR)

(21) Appl. No.: 17/789,749

(22) PCT Filed: Jan. 21, 2021
(86) PCT No.: PCT/EP2021/051394

§ 371 Date: Jun. 28, 2022

(30) Foreign Application Priority Data
Jan. 22, 2020 (EP) 20305051.3

(51) Int. Cl. A61K 8/73; A61K 8/34; A61Q 19/08; A61K 31/728; A61K 47/38; A61K 47/10; A61K 47/26

(52) U.S. Cl. CPC A61K 8/735; A61K 8/731; A61K 8/345; A61Q 19/08; A61K 31/728; A61K 47/38; A61K 47/10; A61K 47/26

(57) ABSTRACT

The invention relates to a composition comprising at least hyaluronic acid and at least one polyol and/or carboxymethyl cellulose (CMC).

The invention also relates to the use of at least one polyol and/or carboxymethyl cellulose to stabilize hyaluronic acid in a composition.

Hyaluronic acid (HA) is a high molecular weight glycosaminoglycan (GAG) consisting of a disaccharide repetition of N-acetylglucosamine and glucuronic acid.

Its high molecular weight may range from 10⁵ to 10⁷ Daltons and the polymer may extend over a length of 2 to 25 μm.

In the rat, half of the hyaluronic acid is found in the skin, and one quarter comes from the skeleton and joints. The rest of the hyaluronic acid is found in the muscles and viscera (Table 1, according to Reed et al. (Reed, 1988))

TABLE 1

| | | total hyaluronic | hyaluronic |
|---|---|---|---|
| | Weight (g) | acid (mg) | acid (%) |
| Whole rat | 200 | 60.5 | 100 |
| Skin | 40.2 | 33.8 | 56 |
| Muscles | 35.7 | 4.69 | 8 |
| Skeleton and supporting | 57.6 | 16.2 | 27 |
| tissues | | | |
| Intestines and stomach | 15.8 | 0.5 | 1 |
| Remaining internal organs | 43.4 | 5.25 | 9 |

In humans in particular, hyaluronic acid is mainly present in the extracellular matrix. Its biological functions include viscoelasticity of liquid connective tissue such as the synovial joint and vitreous fluid of the eye, control of tissue hydration and water transport, supramolecular assembly of extracellular matrix proteoglycans, and receptor-mediated roles in cell detachment, mitosis, migration, tumor development and metastasis, and inflammation.

Studies on healing show that hyaluronic acid is involved in the regulation of inflammation, increases the proliferation of fibroblasts and keratinocytes and collagen synthesis.

In adult skin, hyaluronic acid is degraded into fragments of different sizes by the enzyme activity of hyaluronidases and reactive oxygen and nitrogen species (ROS/RNS). These fragments may stimulate key aspects of wound repair such as wound contraction, inflammation, neoangiogenesis, fibroplasia, myofibroblast differentiation and increased collagen production/cross-linking.

The biological properties of hyaluronic acid depend on its size. As a matter of fact, small fragments of hyaluronic acid will stimulate angiogenesis whereas high molecular weight hyaluronic acid inhibits this. High molecular weight hyaluronic acid will promote the differentiation of monocytes into fibrocytes while low molecular weight hyaluronic acid will inhibit this. Low molecular weight hyaluronic acid improves the skin's self-defense against microorganisms by inducing the release of β-defensin 2 by keratinocytes. Lastly, intermediate molecular weight hyaluronic acid (250 kDa) promotes wound healing in old-age mice. This improvement in wound healing with 250 kDa hyaluronic acid involves an increase in the expression of the mRNA of hyaluronic acid receptors (CD44 and RHAMM), as well as type I and type III collagen.

While other glycosaminoglycans are covalently attached to a protein chain in the Golgi apparatus, hyaluronic acid is synthesized in a very unique way at the inner face of the cell membrane, with the nascent polymer being extruded through the membrane to the outside as it elongates by alternate addition of a glucuronic acid and an N-acetylglucosamine unit. This mode of synthesis therefore enables unrestricted growth of the polymer which could not proceed in the Golgi or endoplasmic reticulum without destroying the cell due to its size. A multigenic family of enzymes, the hyaluronan synthases (HAS) are responsible for its synthesis. The three HAS differ in their temporal expression during development, in their specific activity and in the size of the hyaluronic acid polymers they generate. Hyaluronic acid is synthesized by mesenchymal, epithelial and immune cells as well as by mesenchymal and hematopoietic stem cells.

The fast renewal of hyaluronic acid is due, in part, to its drainage from the tissues from which it is produced to the lymphatic vessels where about 85% is degraded. In structurally dense tissues such as the skeleton and cartilage, it is likely that most of the renewal of hyaluronic acid occurs by metabolic degradation in situ. In the skin and joints, 20-30% of hyaluronic acid renewal occurs by local metabolism and the remainder is eliminated by the lymphatic system. The half-life of the hyaluronic acid is between half a day and 2-3 days independently of the ways in which it is eliminated. In the bloodstream, about 85-90% is eliminated by the liver and 10% by the kidneys which excrete only 1-2% in the urine.

Hyaluronic acid plays an important role first of all in tissue homeostasis and biomechanical integrity due to its remarkable hydrodynamic characteristics, particularly its viscosity and its capacity to retain water.

Hyaluronic acid also enables interaction with proteoglycans and other macromolecules of the extracellular and pericellular matrix. It interacts with the cell surface either directly via specific receptors (among which are CD44, RHAMM (Receptor for Hyaluronic-Acid-Mediated Mobility) and LYVE-1 (Lymphatic Vascular Endothelial Hyaluronan Receptor)) or indirectly through the interaction of these receptors with other membrane receptors.

Signal transduction induced by stimulation of CD44 with hyaluronic acid plays a role:
In cell proliferation and migration, hence its interest in skin healing, but also in tumor proliferation
In angiogenesis, which, depending on the size of the hyaluronic acid, will be stimulated (low molecular weight hyaluronic acid or oligo hyaluronic acid) or inhibited (high molecular weight hyaluronic acid).
In the regulation of inflammation (recruitment of inflammatory cells and secretion of cytokines) via its interaction with Toll-Like Receptors (TLR) 4, TLR2 and CD44

Hyaluronic acid injections into the dermis have been shown to stimulate de novo synthesis of extracellular matrix components. For example, in atrophic skin, hyaluronic acid treatment increases collagen and elastin expression. Similarly, the injection of hyaluronic acid into the dermis of elderly patients stimulates the synthesis of type I collagen, but not in young patients whose skin is not subject to photoaging.

The skin is the main reservoir of hyaluronic acid in the body (Table 1).

The amount of hyaluronic acid in the dermis is very substantially greater than in the epidermis and represents about 50% of the total hyaluronic acid in the body (Table 1). The papillary dermis is richer than the reticular compartment indicating that the fibroblast of the papillary dermis has a high capacity of synthesis of hyaluronic acid, similar to that of synovial fibroblasts.

However, it is interesting to note that almost all of the hyaluronic acid has disappeared from the epidermis in senile skin whereas it persists in aged dermis suggesting that the regulation of its homeostasis depends on different mechanisms in the dermis and the epidermis. However, although the total level of hyaluronic acid in the dermis may remain relatively constant with age, its quality changes. The size of the polymer decreases and it becomes less extractable, suggesting a stronger association with tissue structures and perhaps with another repertoire of hyaladherins. These qualitative alterations could be responsible for the loss of hydration observed in senescent skin. Furthermore, while short exposure to UV transiently induces increased hyaluronic acid deposition and a slight oedematous reaction, repeated exposure to UV triggers a wound repair type response. The GAGs found in photoaged skin are similar to those present in scar tissue, with a reduced proportion of hyaluronic acid in favor of chondroitin sulfate-rich proteoglycans. Free radicals generated by UV-B could destroy hyaluronic acid polymers and generate biologically active, pro-inflammatory and pro-angiogenic fragments. The level of hyaluronic acid synthesis is now easily monitored by measuring the expression of HAS genes present in the skin. Their expression is stimulated by TGFβ in both the dermis and the epidermis, but with different kinetics. Other growth factors, such as PDGF, also have a stimulating activity. However, HAS expression, and thus hyaluronic acid production, is almost completely excluded by glucocorticoids.

It is understood from the above that the activity of hyaluronic acid is a function of the size of its fragments once degraded, with high molecular weight hyaluronic acid having a better activity than low molecular weight hyaluronic acid.

It is known to use Hyaluronic acid in cosmetic or dermatological compositions for topical application.

However, hyaluronic acid has a degree of instability. Thus, when it is introduced into a cosmetic composition, its effectiveness decreases over time due to its degradation. Moreover, the composition to which it is added, after a certain time of storage, presents signs of degradation: coloration, odor, which are unacceptable for the user.

It could therefore be advantageous to have a composition, particularly a cosmetic and/or pharmaceutical composition, in which hyaluronic acid could be protected, at least partially, from degradation, so that the hyaluronic acid contained in the said composition has good level of activity.

This is one of the objects of the present invention.

Indeed, the applicant has now discovered that surprisingly, after long and laborious work, polyols and/or carboxymethylcellulose (CMC) could have a protective effect on hyaluronic acid, thus making it possible to avoid its degradation and maintaining increased stability over time, which is a guarantee of a better activity over a long period by avoiding, or at least slowing down, its degradation.

Thus, a primary object of the invention is a composition, advantageously a cosmetic or pharmaceutical composition, comprising at least hyaluronic acid and at least one polyol and/or carboxymethyl cellulose (CMC).

Preferably the composition according to the invention may comprise, in addition to hyaluronic acid, at least one polyol and carboxymethyl cellulose.

According to the invention, the hyaluronic acid may be a hyaluronic acid with a molecular weight comprised between 10⁵ and 10⁷, preferably between 10⁵ and 4.10⁶ and very preferably between 5.10⁵ and 2.10⁶ Da.

By polyol is meant a saturated or unsaturated, linear, branched or cyclic alkyl compound with at least two —OH functions on the alkyl chain, as well as polymers (polyethers) of these polyhydroxylated alkyl compounds. Preferably it is an alkyl compound having from 2 to 12 carbon atoms, and still more preferably from 2 to 8 carbon atoms. Advantageously, this alkyl compound has 2 or 3 carbon atoms. According to the invention, the hyaluronic acid may be present in the composition in an amount comprised between 0.01% and 20% of the total weight of the composition, preferably between 0.03% and 10% of the total weight of the composition, very preferably between 0.05% and 1% of the total weight of the composition.

According to the invention the polyol may be selected from ethylene glycol [(HOCH₂—CH₂OH)], diethylene glycol [(HOCH₂—CH₂—O—CH₂—CH₂OH)], triethylene glycol [(HOCH₂—CH₂—O—CH₂—CH₂—CH₂—CH₂OH], propylene glycol [(propane-1,2-diol: HOCH₂—CHOH—CH₃)], trimethylene glycol [(propane-1,3-diol: HOCH₂—CH₂—CH₂OH)], propylene glycol, polymers and copolymers of glycerol, ethylene glycol and propylene glycol, for example such as dipropylene glycol and hexaglycerol hexylene glycol, pentylene glycol, butyldiglycol, 1,2,3trihydroxyhexane, butylene glycol [(butane-1,3-diol)], n-butylene glycol [(butane-1,4-diol)], 2,3-butylene glycol [or secbutylene glycol (butane-2,3-diol)], or Triols, for example Glycerol; Tetraols, for example such as erythritol, threitol; pentols (pentanols), for example such as xylitol, arabitol (lyxitol), ribitol (adonitol); hexols, for example such as sorbitol (gulitol), dulcitol (galactitol), mannitol, fucitol, iditol; heptols for example such as volemitol; or isomalt, maltitol, isomaltitol, lactitol (lactositol), maltotriitol, maltotetraitol, polyglycitol.

Preferably according to the invention, the polyol may be selected from triols and hexols, very preferably from glycerol, sorbitol and mannitol.

Also according to the invention, the polyol may be present in the composition in an amount comprised between 0.05% and 90% of the total weight of the composition, preferably between 0.5% and 80% of the total weight of the composition, very preferably between 1.0 and 75% of the total weight of the composition.

According to the invention, the carboxymethyl cellulose may be present in the composition in an amount comprised between 0.1% and 72% of the total weight of the composition, preferably between 0.5% and 50% of the total weight of the composition, very preferably between 1 and 5% of the total weight of the composition.

Still according to the invention, the ratio between the hyaluronic acid and the polyol in the composition may be comprised between 0.0001 and 400, preferably between 0.0003 and 2, very preferably between 0.0006 and 1.

Also according to the invention, the ratio between the hyaluronic acid and the carboxymethyl cellulose in the composition may be comprised between 0.0001 and 200, preferably between 0,0006 and 20, and very preferably between 0.01 and 1.

Still according to the invention, the ratio in the composition between the polyol and the carboxymethyl cellulose may be comprised between 0.0007 and 900, preferably between 0.0001 and 160, very preferably between 0.2 and 75.

According to a second object, the invention relates to the use of at least one polyol and/or carboxymethyl cellulose to slow down, limit or even eliminate the degradation of hyaluronic acid, advantageously in a composition, particularly a cosmetic or pharmaceutical composition.

According to a third object, the invention relates to the use of at least one polyol and/or carboxymethyl cellulose to slow down, limit or even annihilate, advantageously in a composition, particularly a cosmetic or pharmaceutical composition, the degradation of hyaluronic acid induced by ionizing radiation for example such as radiation of the beta or gamma types or ultraviolet radiation.

According to a fourth object, the invention relates to the use of at least one polyol and/or carboxymethyl cellulose to slow down, limit or even eliminate the degradation of hyaluronic acid induced by oxidative stress, advantageously in a composition, particularly a cosmetic or pharmaceutical composition.

According to a fifth object, the invention relates to the use of at least one polyol and/or carboxymethyl cellulose to slow down, limit or even eliminate the degradation of hyaluronic acid present in a composition provided to promote wound healing.

According to a sixth object, the invention relates to the use of at least one polyol and/or carboxymethyl cellulose to slow down, limit or even eliminate the degradation of hyaluronic acid present in a composition provided to modulate inflammation.

According to a seventh object, the invention relates to the use of at least one polyol and/or carboxymethyl cellulose to slow down, limit or even eliminate the degradation of hyaluronic acid present in a composition provided to increase the proliferation of fibroblasts and/or keratinocytes.

According to an eighth object, the invention relates to the use of at least one polyol and/or carboxymethyl cellulose to slow down, limit or even eliminate the degradation of hyaluronic acid present in a composition provided to stimulate the synthesis of collagen.

According to a ninth object, the invention relates to the use of at least one polyol and/or carboxymethyl cellulose to slow down, limit or even eliminate the degradation of hyaluronic acid present in a composition provided to inhibit angiogenesis.

According to a tenth object, the invention relates to the use of at least one polyol and/or carboxymethyl cellulose to slow down, limit or even eliminate the degradation of hyaluronic acid present in a composition provided to promote the differentiation of monocytes into fibrocytes.

According to an eleventh object, the invention relates to the use of at least one polyol and/or carboxymethyl cellulose to slow down, limit or even eliminate the degradation of hyaluronic acid present in a composition provided to treat skin ageing.

According to a twelfth object, the invention relates to the use of at least one polyol and/or carboxymethyl cellulose to slow down, limit or even eliminate the degradation of hyaluronic acid present in a composition provided to treat wrinkles, particularly in filling the wrinkles.

According to a thirteenth object, the invention relates to the use of at least one polyol and/or carboxymethyl cellulose to slow down, limit or even eliminate the degradation of hyaluronic acid present in a composition provided to be used in mesotherapy, particularly in the rehydration/rejuvenation of tissues.

It can thus be understood that according to the invention the composition may comprise, in addition to hyaluronic acid, at least one polyol alone or at least carboxymethyl cellulose alone or a mixture of at least one polyol and carboxymethyl cellulose. Preferably according to the invention the composition may comprise a mixture of at least one polyol and carboxymethyl cellulose.

The composition of the invention may take the form of all known galenic forms, advantageously for topical application, in particular in the form of an aqueous, hydroalcoholic or oily solution, an oil-in-water or water-in-oil emulsion or a multiple emulsion, an aqueous or oily gel, a liquid, pasty or solid anhydrous product, a dispersion of oil in an aqueous phase by means of spherules, these spherules being able to be polymeric nanoparticles such as nanospheres and nanocapsules or lipid vesicles of ionic and/or non-ionic type.

This composition may be fluid to a greater or lesser extent and have the aspect of a white or colored cream, an ointment, a milk, a lotion, a serum, a paste, a foam. It may possibly be applied to the skin or hair in the form of an aerosol. It may also be in solid form, for example in the form of a stick. It may be used as a care product and/or as a make-up product. It may also be in the form of shampoos or conditioners.

In known manner without limitation, the composition of the invention may contain the adjuvants that are usual in the cosmetic and dermatological fields, such as hydrophilic or lipophilic gelling agents, hydrophilic or lipophilic active ingredients, preservatives, antioxidants, organic or inorganic solvents, perfumes, fillers, filters, pigments, odor absorbers and colorants, fatty substances, ionic or non-ionic thickeners, softeners, anti-free radical agents, opacifiers, stabilizers, emollients, silicones, α-hydroxy acids, anti-foaming agents, moisturizing agents, vitamins, surfactants, sequestrants, polymers, propellants; Alkalizing or acidifying agents, or any other ingredients commonly used in the field.

The amounts of these various adjuvants may be those conventionally used in the fields considered for example from 0.001 to 90%, preferably from 1.0% to 75%, of the total weight of the composition. These adjuvants, depending on their nature, may be added to the fatty phase, to the aqueous phase, to the lipid vesicles and/or to the nanoparticles.

The fatty substances may be constituted by an oil or a wax or mixtures thereof. By oil, is meant a compound which is liquid at room temperature. By wax, is meant a compound which is solid or substantially solid at ambient temperature, and whose melting point is generally higher than 35° C.

As oils, there may be mentioned mineral oils (petroleum jelly); vegetable oils (sweet almond oil, macadamia oil, blackcurrant seed oil, jojoba oil); synthetic oils such as perhydrosqualene, alcohols, fatty esters or acids (such as the benzoate of C12-C15 alcohols sold under the trade name “Finsolv TN” by the Finetex company, octyl palmitate, isopropyl lanolate, triglycerides among which are those of capric/caprylic acids), oxyethylenated or oxypropylenated fatty esters and ethers; those that are silicosed (cyclomethicone, polydimethysiloxanes or PDMS) or fluorinated, polyalkylenes.

As waxy components, there may be mentioned paraffin, carnauba wax, beeswax, hydrogenated castor oil.

Among the organic solvents, the alcohols may be mentioned.

The thickeners may be chosen in particular from cross-linked polyacrylic acids, modified or unmodified guar gums and celluloses such as hydroxypropylated guar gum, methylhydroxyethyl cellulose and hydroxypropyl methyl cellulose, and skin-colouring agents, for example such as mono- or polycarbonyl derivatives such as isatin, alloxane, ninhydrin, glyceraldehyde and mesotartaric aldehyde, Of course, the person skilled in the art will take care to choose the above-mentioned possible complementary component(s) and/or their quantities in such a way that the advantageous properties intrinsically attached to the use of hyaluronic acid in accordance with the invention are not, or are substantially not, altered by the envisioned addition or additions. The compositions according to the invention may be prepared according to techniques well known to the person skilled in the art, in particular those intended for the preparation of oil-in-water (O/W) or water-in-oil (W/O) type emulsions. This composition may in particular take the form of a simple or complex emulsion (O/W, W/O, O/W/O or W/O/W) such as a cream, a milk, or in the form of a gel or a cream gel, in the form of a lotion, a powder, a solid stick and optionally be packaged as an aerosol and be in the form of a foam or spray. Preferably, the compositions according to the invention are in the form of an oil-in-water emulsion.

When it is an emulsion, the aqueous phase thereof may comprise a nonionic vesicular dispersion prepared according to known procedures (Bangham, Standish and Watkins. J. Mol. Biol. 13, 238 (1965), FR 2 315 991 and FR 2 416 008).

Other features of the invention may be apparent from the following specific, but not limiting, examples illustrating the invention, in which

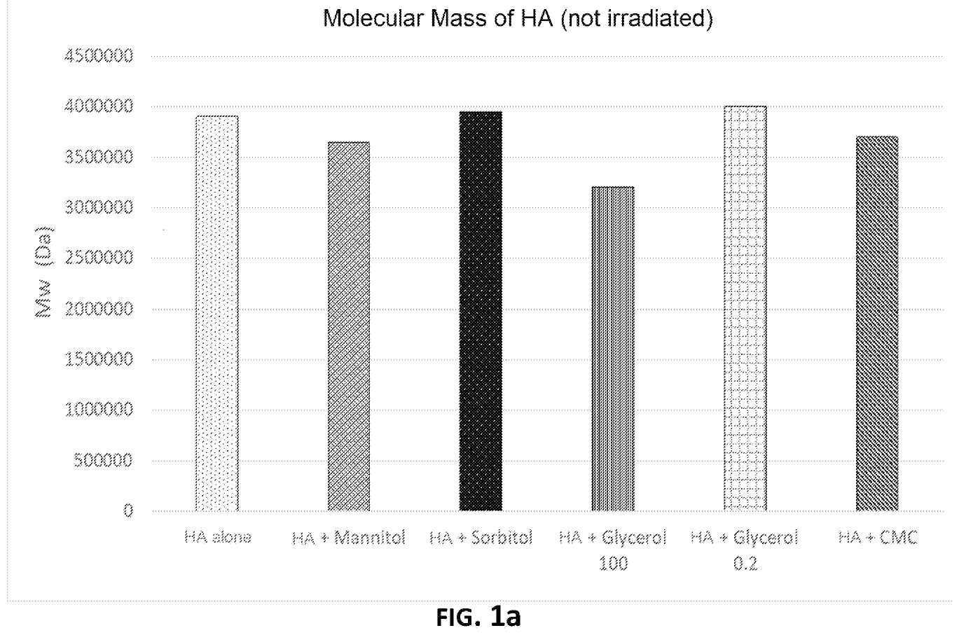

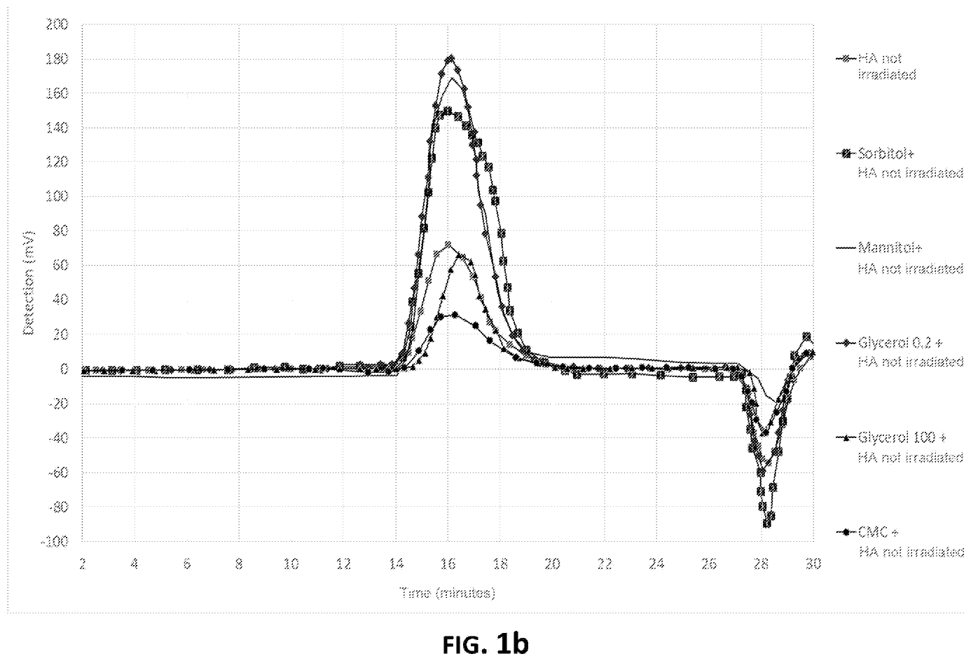

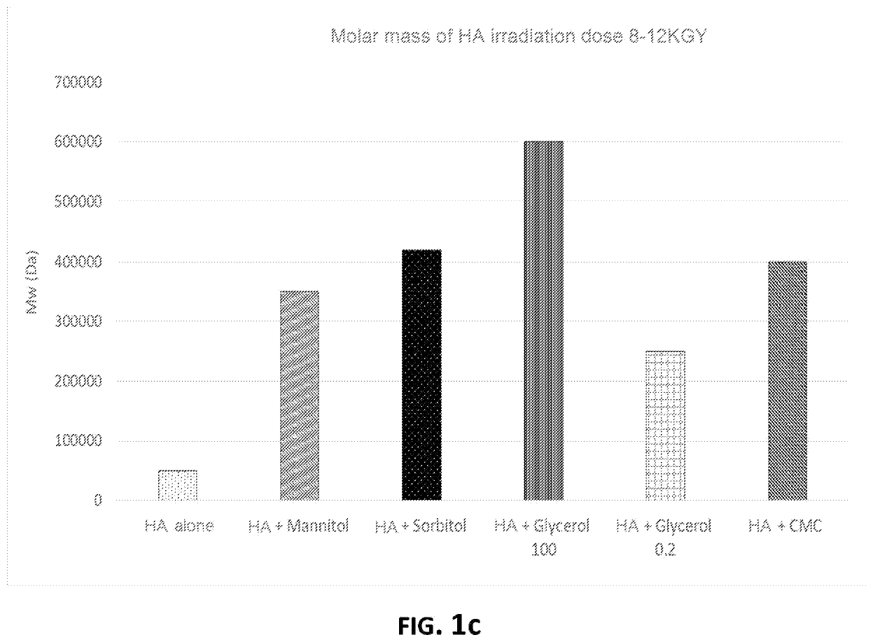

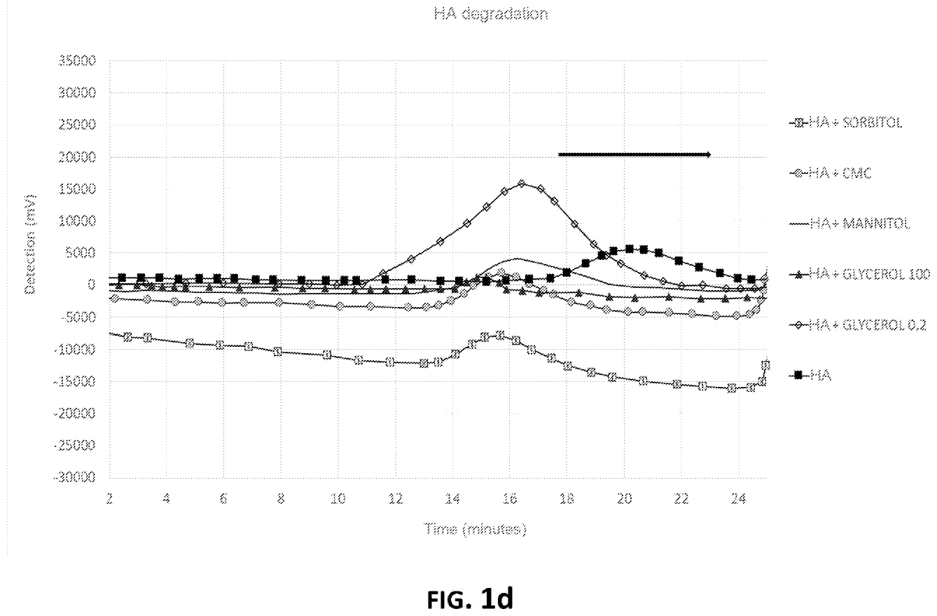

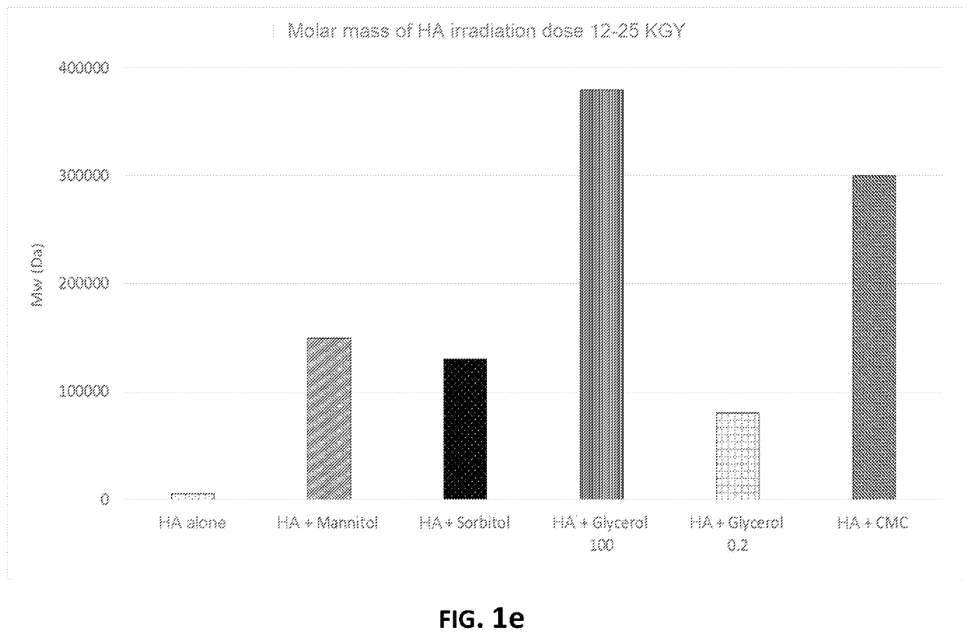

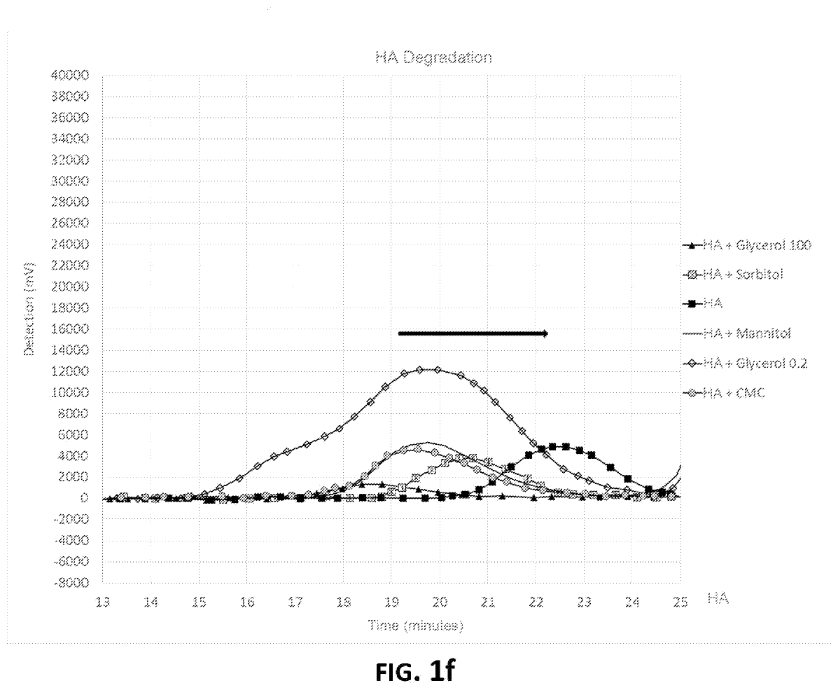

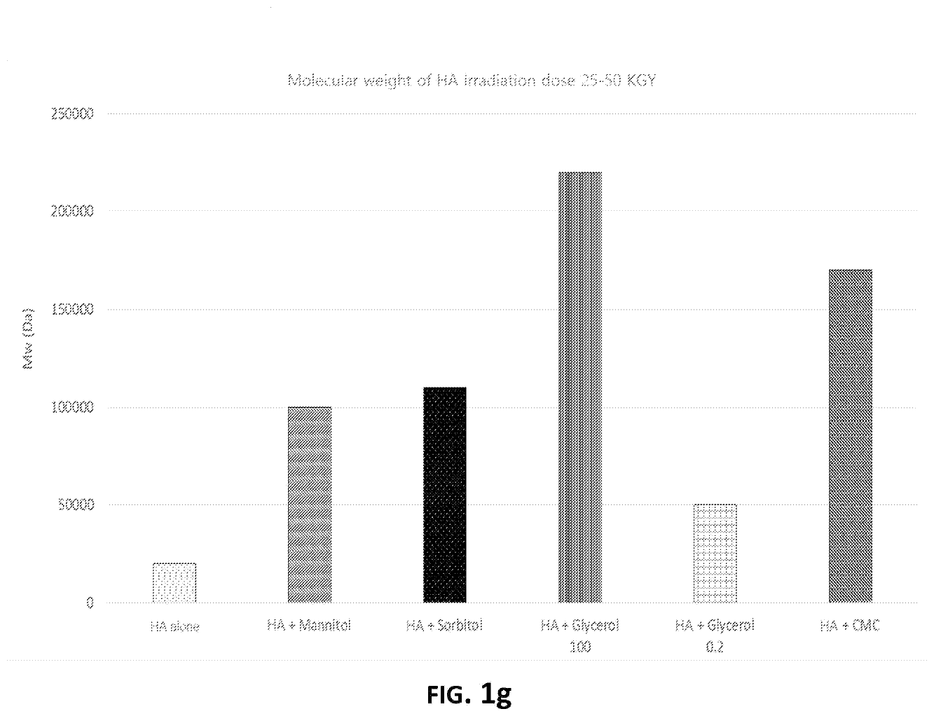

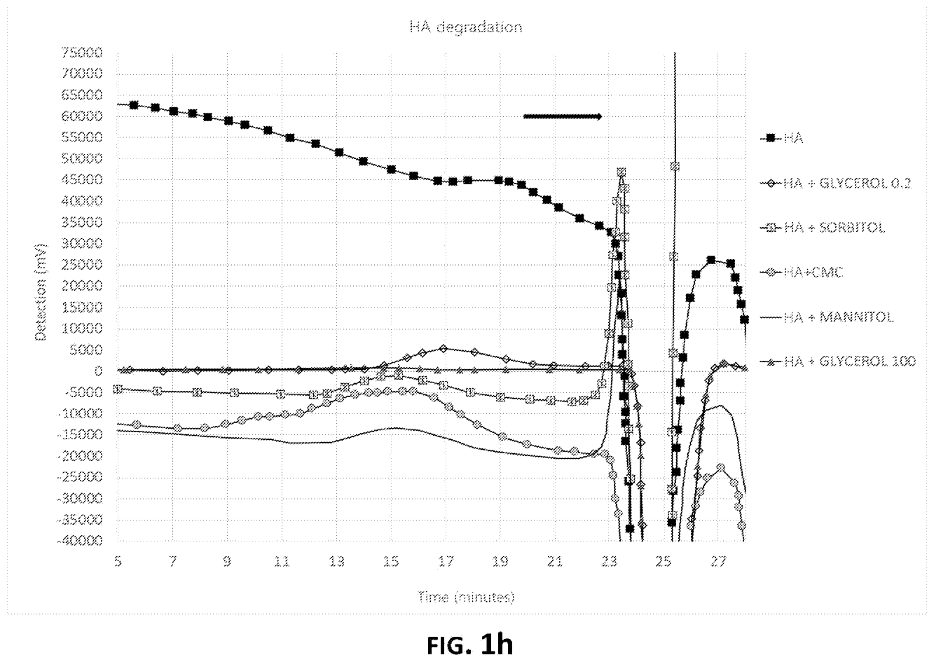

FIG. 1 presents the results obtained during the study by gel permeation chromatography (GPC) of the degradation of hyaluronic acid in compositions comprising a polyol and carboxymethyl cellulose (CMC), subjected or not subjected to a stress of ionizing radiation type.

FIG. 1a presents the results of GPC analysis of hyaluronic acid degradation in non-irradiated compositions.

FIG. 1b presents the molar masses (MW) of HA as a function of their association with polyols like mannitol, sorbitol, glycerol and CMC.

FIG. 1c presents the results of the analysis of hyaluronic acid degradation under stress conditions induced by an eBeam irradiation dose of 8 to 12 kGy that was or was not previously associated with different polyols or CMC.

FIG. 1d presents the change in the molar masses (MW) of HA, under the stress condition induced by an eBeam irradiation dose of 8 to 12 kGy and as a function of their association with the polyols of type mannitol, sorbitol, glycerol and CMC. The arrow indicates a shift towards lower molecular weight (higher retention time).

FIG. 1e presents the results of the hyaluronic acid degradation analysis under eBeam irradiation conditions at 12-25 kGy.

FIG. 1f presents the change in the molar masses (MW) of HA, under eBeam irradiation stress conditions at a dose of 12 to 25 kGy and as a function of their association with polyols of type mannitol, sorbitol, glycerol and CMC. The arrow indicates a shift towards lower molecular weight (higher retention time).

FIG. 1g presents the results of the hyaluronic acid degradation analysis under eBeam irradiation conditions at 25-50 kGy.

FIG. 1h presents the change in the molar masses (MW) of HA, under eBeam irradiation stress conditions at a dose of 25 to 50 kGy and as a function of their association with polyols of type mannitol, sorbitol, glycerol and CMC. The arrow indicates a shift towards lower molecular weight (higher retention time).

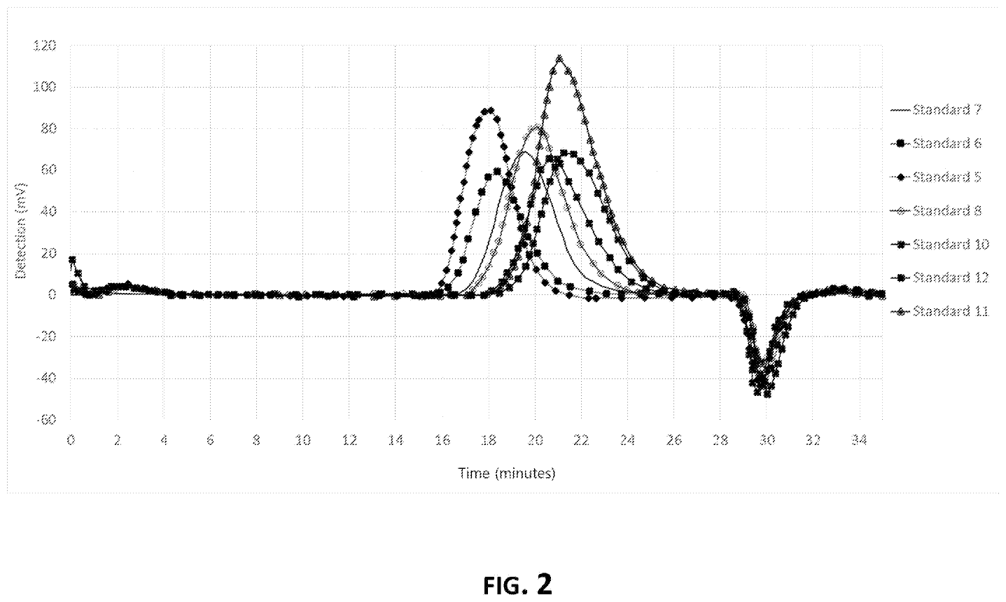

FIG. 2 presents the elution profiles of the reference hyaluronic acid standards in the context of the effect of oxidative stress.

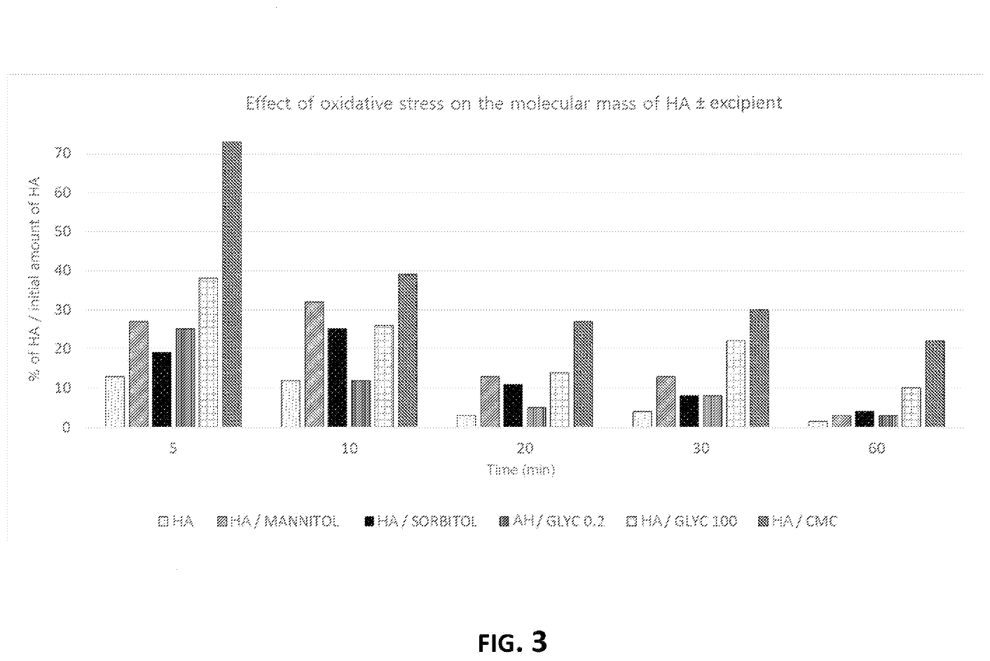

FIG. 3 prevents the effects of oxidative stress on the molecular weight of hyaluronic acid in the presence or absence of polyols as a function of time.

### EXAMPLE 1: COMPARATIVE EVALUATION OF THE EFFECT OF EXCIPIENTS ON HYALURONIC ACID BEFORE AND AFTER IRRADIATION

The aim of this experimentation is to evaluate comparatively the molecular weight of hyaluronic acid dissolved in different excipient solutions, before and after irradiation at different doses.
1. Principle

Hyaluronic acid is dissolved in different solutions of excipients to study their effect on its molecular weight before and after Ebeam irradiation at three different doses (8-12 kGy, 12-25 kGy and 25-50 kGy).
2. Materials and Methods
   a. Materials
      5 mL glass tubes+stoppers+metal caps
      1 mL GPC vials (Agilent ref 5188-6593)
      Hyaluronic acid GPC filter (PTFE 13 mm 0.2 μm Agilent 5190 5265)
      Sartorius Scale (No. 4)
   b. Reagents
      Hyaluronic acid (Altergon batch 9998-3M Da)
      Mannitol (Merck batch FN 1378803750)
      Sorbitol (Roquette batch E090B)
      Glycerol (Sigma batch SHBK3676)
      CMC (Coluxia batch C161437)
      Distilled water
      Ultra pure water GPC grade
      NaCl (Fisher ref 10274392)
      Methanol (Fisher 1851587)
      Ultra pure water GPC grade (Merk ref 1.115333.1000)
   c. Preparation of the Solutions

The set of solutions is prepared according to the following table:

| hyaluronic | Glycerol | Mannitol | Sorbitol | CMC | Volume distilled |
|---|---|---|---|---|---|
| acid (mg) | (g) | (mg) | (mg) | (mg) | water (mL) |
| 200 | — | 1050 | — | — | Qsp 20 |
| 200 | — | — | 1050.00 | — | |
| 200 | 100 | — | — | — | |
| 200 | 0.2 | — | — | — | |
| 200 | — | — | — | 1000 | |
| 200 | — | — | — | — | |

The solutions thus prepared are then freeze-dried (Cryotec Pilot Compact) according to the protocol comprising a first step of freezing by passing from a temperature of +25° C. to a temperature of −45° C. in 60 minutes, then maintaining at −45° C. for 6 hours; a second step of sublimation by passing from a temperature of −45° C. to a temperature of −20° C. (under a vacuum of 0.16 mBar), for 4 hours, then maintaining at −20° C. for 24 hours (under a vacuum of 0.16 mBar); a third step of secondary drying by passing from a temperature of 20° C. to 25° C. in 4 hours (under a vacuum of 0.007 mBar) and maintaining at the temperature of +25° C. for 15 hours (under a vacuum of 0.007 mBar).

After freeze-drying, the samples are then subjected to Beta irradiation in a Mevex A29, 34 kW, 10 Mev apparatus at a frequency of 640 Hz, with a scan setting of 2.7A, for a number of revolutions, at a speed of 2.51 m/min, at the following doses: 0, 8-12, 17.5-25 and 25-50 kGy.
3. GPC Analysis
   3.a Preparation of Samples for Hyaluronic Acid Analysis

Before and after irradiation, each prepared solution is analyzed with GPC according to the following protocol:

A dilution in a mixture of 2M NaCl/MeOH 2% is carried out if necessary in order to obtain a final concentration of hyaluronic acid between 0.5 and 1 g/L.

The samples are then filtered and dispensed into vials for analysis with GPC.
3.b Analysis of the Hyaluronic Acid:

The analysis of the molecular mass of the hyaluronic acid (between 64 kDa and 1.3 MDa) was performed in an Agilent SL1200 apparatus according to the following protocol:

Columns: TSKgel GMPWXL and associated guard column;

Column/detector temperature: 45° C.;

Flow rate: 0.5 mL/min 0.2 M NaCl and 2% MeOH;

Detectors:

Refractive Index Detector (RID)→Temperature: 45° C.

Diode Array Detectors (DAD)→Wavelength: 205 nm

Injection volume: 50 μL
3.c Results of GPC Analysis
   3.c.1 Range
      The following reference range is established.

| | Retention time | | | |
|---|---|---|---|---|
| | at the apex of | Molecular | | |
| | the peak | weight | Log | |
| Point | (mins) | (MW) | MW | % error |
| 1 | 15.80940 | 1300000 | 6.11 | −29.42 |
| 2 | 17.16300 | 720000 | 5.86 | 21.25 |
| 3 | 17.82000 | 390000 | 5.59 | 14.25 |
| 4 | 17.82000 | 1 | 0.00 | −33440718.57 |
| 5 | 18.79920 | 210000 | 5.32 | 27.50 |
| 6 | 19.07820 | 90000 | 4.95 | −35.19 |
| 7 | 19.57320 | 77000 | 4.89 | −6.16 |
| 8 | 19.83960 | 60000 | 4.78 | −9.98 |

The coefficient for determining R2 has the value 0.958192; the range is validated.
3.c.2 Samples

The table below shows the results obtained on the samples tested.

| | | Molar | Molar | | |
|---|---|---|---|---|---|
| | | mass at | mass | Molar | |
| | | the | as a | mass in | |
| | Irradiation | Apex | number | mass | Polydispersity |
| Samples | dose | Mp | Mn | Mw | PD |
| hyaluronic acid alone | not | 3955260 | 1889602 | 3956345 | 2.1 |
| | irradiated | | | | |
| | 8-12 kGy | 324438 | 27570 | 35356 | 1.28 |
| | 12-25 kGy | 14577 | 8629 | 19307 | 2.2 |
| | 25-50 kGy | 19327 | 8117 | 14915 | 1.84 |
| hyaluronic acid + | not | 3792786 | 2045056 | 3555105 | 1.74 |
| Mannitol | irradiated | | | | |
| | 8-12 kGy | 337323 | 246524 | 340051 | 1.38 |
| | 12-25 kGy | 174929 | 71073 | 165084 | 2.32 |
| | 25-50 kGy | 91903 | 72292 | 100650 | 1.4 |
| hyaluronic acid + | not | 4276571 | 2411778 | 3995109 | 1.66 |
| Sorbitol | irradiated | | | | |
| | 8-12 kGy | 266860 | 218014 | 408302 | 1.87 |
| | 12-25 kGy | 145154 | 78841 | 152422 | 1.93 |
| | 25-50 kGy | 60243 | 40529 | 109265 | 2.7 |
| hyaluronic acid + | not | 2991852 | 2089073 | 3077895 | 1.47 |
| Glycerol 100 | irradiated | | | | |
| | 8-12 kGy | 502827 | 297841 | 598552 | 2.01 |
| | 12-25 kGy | 379800 | 224544 | 394409 | 1.76 |
| | 25-50 kGy | 235647 | 101000 | 215774 | 2.14 |
| hyaluronic acid + | not | 3898463 | 2400824 | 4003304 | 1.67 |
| Glycerol 0.2 | irradiated | | | | |
| | 8-12 kGy | 278292 | 110160 | 245240 | 2.2 |
| | 12-25 kGy | 71869 | 39110 | 79587 | 2.04 |
| | 25-50 kGy | 44015 | 22988 | 49331 | 2.15 |
| hyaluronic acid + | not | 4053720 | 1851695 | 3635442 | 1.96 |
| CMC | irradiated | | | | |
| | 8-12 kGy | 200695 | 104860 | 404981 | 3.86 |
| | 12-25 kGy | 85739 | 50251 | 300369 | 5.9 |
| | 25-50 kGy | 82814 | 55613 | 172783 | 3.1 |

These results are also presented in FIGS. 1a to 1f
4. Conclusions

It is observed that:
Whatever the solution in which the hyaluronic acid is put back into suspension, prior to irradiation, its molecular weight remains stable.
The higher the dose of irradiation, the more the hyaluronic acid is degraded.
A protective effect by each excipient on the hyaluronic acid during irradiation, whatever the dose, the greatest protective effect being observed in the presence of glycerol 100 and CMC.

### EXAMPLE 2: COMPARATIVE EVALUATION OF THE EFFECT OF EXCIPIENTS ON HYALURONIC ACID IN AND NOT IN A CONDITION OF OXIDATIVE STRESS

The aim of this experiment is to evaluate comparatively the effect of oxidative stress on the molecular weight of the hyaluronic acid dissolved in different excipient solutions.
1. Principle

The hyaluronic acid is dissolved in different excipient solutions to study their effect on its molecular weight in the presence or absence of hydrogen peroxide and copper chloride according to the protocol of Chen et al. 2019 (Molecules 2019, 24, 61).
2. Materials and methods
   a. Materials
      5 mL glass tubes+stoppers+metal caps
      1 mL GPC vials (Agilent ref 5188-6593)
      Hyaluronic acid GPC filter (PTFE 13 mm 0.2 μm Agilent 5190 5265)
      Water bath (Memmert 10 L)
      Sartorius Scale (No. 4)
   b. Reagents
      Hyaluronic acid (Altergon batch 9998-3M Da)
      Mannitol (Merck Batch FN 1378803750)
      Sorbitol (Roquette batch E090B)
      Glycerol (Sigma batch SHBK3676)
      CMC (Coluxia batch C161437)
      H₂O₂(Fisher ref 15632040)
      CuCl₂ (Fisher ref 10093650)
      Distilled water
      Ultra pure water GPC grade
      NaCl (Fisher ref 10274392)
      Methanol (Fisher 1851587)
      Sodium Chloride (NaCl)
      Ultra pure water GPC grade (Merk ref 1.115333.1000)
   c Preparation of the solutions
      NaCl Solution 0.2M:
      The molecular weight of the NaCl being 58.44 g·mol⁻¹, a 1.17% solution is prepared, i.e. 2.34 g in 200 mL of distilled water.
      H₂O₂ solution at 3%: the 30% H₂O₂ stock solution is diluted by a factor of 10.
      CuC12 solution at 50 mM:
      The molecular weight of CuCl₂ being 170.48 g·mol⁻¹, 8.5 mg is weighed into 20 mL of distilled water.
      Sample Preparations:
      All the solutions were prepared according to the following table to obtain a hyaluronic acid concentration of 0.5% w/v according to the protocol of Chen eta/2019:

| Hyaluronic acid | Glycerol | Mannitol | Sorbitol | CMC | NaCl 0.2M |
|---|---|---|---|---|---|
| (mg) | (g) | (mg) | (mg) | (mg) | (mL) |
| 100 | — | 525 | — | — | Qsp 20 |
| 100 | — | — | 525 | — | |
| 100 | 50 | — | — | — | |
| 100 | 0.1 | — | — | — | |
| 100 | — | — | — | 500 | |
| 100 | — | — | — | — | |

3. Oxidative Stress Protocol

Two 1 mL tubes are prepared for each sample and each time: 1 without addition, 1 to which are added 5 mM of H₂O₂ i.e. 5.7 μL/tube of the solution of H₂O₂ at 3% and 5 μM of CuCl₂ i.e. 2 μL/tube of the solution prepared previously.

The samples under oxidative stress conditions are incubated at 50° C. for 5, 10, 20, 30 and 60 min.

After each incubation period, the reaction is stopped by quick freezing at −80° C. for GPC analysis.
4. GPC Analysis
   4.a Preparation of samples for hyaluronic acid analysis

### Preparation of the Range of Standards

The standards (1 to 7) and the samples to be analyzed are prepared in 0.2M NaCl/2% MeOH buffer (which serves as eluent) at concentrations between 0.5 and 1 g/L and then filtered (0.22 μM) before being sampled in vials for GPC analysis.

Analysis by GPC (Gel Permeation Chromatography)

The analysis of the molecular mass of the hyaluronic acid (between 64 kDa and 1.3 MDa) was performed in an AGILENT SL 1200 apparatus according to the following protocol:

Columns: TSKgel GMPWXL and associated guard column

Column/detector temperature: 45° C.

Flow rate: 0.5 mL/min 0.2 M NaCl and 2% MeOH

Detectors:

RID→Temperature: 45° C.

DAD→Wavelength: 205 nm

Injection volume: 50 μL
4.b Results of GPC Analysis
   4.b.1 Range
   The following reference range is established.

| | Retention time | | | |
|---|---|---|---|---|
| | at the apex of | Molecular | | |
| | the peak | weight | Log | |
| Standards | (mins) | (MW) | MW | % error |
| 1 | 18.00540 | 1300000 | 6.11 | 12.81 |
| 2 | 18.29880 | 720000 | 5.86 | −22.91 |
| 3 | 19.33920 | 390000 | 5.59 | 5.66 |
| 4 | 20.03220 | 210000 | 5.32 | 2.36 |
| 5 | 20.70720 | 77000 | 4.89 | −50.68 |
| 6 | 21.13920 | 90000 | 4.95 | 10.46 |
| 7 | 21.34260 | 60000 | 4.75 | −13.13 |

Determination coefficient: 0.986936; Coefficient of linear correlation: −0.993446; Standard Y Error E

The standard range is in accordance with an R² of 0.99. The analyses can continue (see FIG. 2)
4.b.2 Sample

The table below shows the results obtained on the samples tested.

| Samples | Time | Mp | Mn | Mw | PD | % |
|---|---|---|---|---|---|---|
| hyaluronic acid | T = 0 | 1736745 | 958442 | 2055581 | 2.1 | 100.0 |
| alone | | | | | | |
| hyaluronic | T = 5 min | 220759 | 59767 | 255328 | 4.3 | 12.4 |
| acid + 5M | T = 10 min | 138791 | 54202 | 232579 | 4.3 | 11.3 |
| H₂O₂/CuCl₂ | T = 20 min | 38177 | 11575 | 46620 | 4 | 2.3 |
| | T = 30 min | 44505 | 18640 | 58951 | 3.2 | 2.9 |
| | T = 60 Min | 7822 | 2847 | 11561 | 4 | 0.6 |
| hyaluronic | T = 0 | 2187646 | 118176 | 2421190 | 2 | 100.0 |
| acid + mannitol | | | | | | |
| hyaluronic | T = 5 min | 542153 | 244225 | 656459 | 2.7 | 27.1 |
| acid + mannitol + | T = 10 min | 546024 | 249815 | 767406 | 3.072 | 31.7 |
| H₂O₂/CuCl₂ | T = 20 min | 117619 | 63292 | 295937 | 4.7 | 12.2 |
| | T = 30 min | 137950 | 62881 | 275914 | 4.4 | 11.4 |
| | T = 60 Min | 45738 | 17750 | 88586 | 5 | 3.7 |
| hyaluronic | T = 0 | 2392690 | 1281982 | 2617233 | 2 | 100.0 |
| acid + sorbitol | | | | | | |
| hyaluronic | T = 5 min | 415459 | 184625 | 503731 | 2.7 | 19.2 |
| acid + sorbitol + | T = 10 min | 416701 | 180666 | 565745 | 3.1 | 21.6 |
| H₂O₂/CuCl₂ | T = 20 min | 106726 | 52670 | 291150 | 5.5 | 11.1 |
| | T = 30 min | 97580 | 43554 | 201977 | 4.64 | 7.7 |
| | T = 60 Min | 34275 | 13963 | 107268 | 7.7 | 4.1 |
| hyaluronic | T = 0 | 2049366 | 1046444 | 2214354 | 2.1 | 100.0 |
| acid + glycerol 0.2 | | | | | | |
| hyaluronic | T = 5 min | 394921 | 185736 | 485579 | 2.6 | 21.9 |
| acid + glycerol 0.2 + | T = 10 min | 197106 | 83178 | 246580 | 2.7 | 11.1 |
| H₂O₂/CuCl₂ | T = 20 min | 76416 | 31075 | 102560 | 3.3 | 4.6 |
| | T = 30 min | 88543 | 35747 | 170932 | 4.9 | 7.7 |
| | T = 60 Min | 29402 | 10610 | 85505 | 8 | 3.9 |
| hyaluronic | T = 0 | 2530971 | 1516634 | 2743329 | 1.8 | 100.0 |
| acid + glycerol 100 | | | | | | |
| hyaluronic | T = 5 min | 961714 | 433316 | 1021851 | 2.4 | 37.2 |
| acid + glycerol 100 + | T = 10 min | 633176 | 308487 | 704977 | 2.3 | 25.7 |
| H₂O₂/CuCl₂ | T = 20 min | 337409 | 162026 | 369177 | 2.3 | 13.5 |
| | T = 30 min | 481365 | 207910 | 596919 | 2.9 | 21.8 |
| | T = 60 Min | 164272 | 88322 | 239735 | 2.7 | 8.7 |
| hyaluronic | T = 0 | 1335506 | 463913 | 1351801 | 2.9 | 100 |
| acid + CMC | | | | | | |
| hyaluronic | T = 5 min | 729745 | 158142 | 987117 | 6.2 | 73.0 |
| acid + CMC + | T = 10 min | 279924 | 73608 | 505860 | 6.9 | 37.4 |
| H₂O₂/CuCl₂ | T = 20 min | 272377 | 51313 | 360989 | 7 | 26.7 |
| | T = 30 min | 260247 | 64023 | 383282 | 6 | 28.4 |
| | T = 60 Min | 171667 | 38554 | 273637 | 7.1 | 20.2 |

The following table shows the percentage of hyaluronic acid in each sample after incubation compared to the initial amount of hyaluronic acid

| Time | HA + 5M | | | HA/glyc | HA/glyc | |
|---|---|---|---|---|---|---|
| (min.) | H₂O₂/CuCl₂ | HA/mannitol | HA/sorbitol | 0, 2 | 100 | HA/CMC |
| 0 | 100 | 100 | 100 | 100 | 100 | 100 |
| 5 | 12.4 | 27.1 | 19.2 | 21.9 | 37.2 | 73 |
| 10 | 11.3 | 31.7 | 21.6 | 11.1 | 25.7 | 37.4 |
| 20 | 2.3 | 12.2 | 11.1 | 4.6 | 13.5 | 26.7 |
| 30 | 2.9 | 11.4 | 7.7 | 7.7 | 21.8 | 28.4 |
| 60 | 0.6 | 3.7 | 4.1 | 3.9 | 8.7 | 20.2 |

HA: Hyaluronic Acid

These results are also presented in FIG. 3.
1. Conclusions

The following is observed:
Almost complete degradation of hyaluronic acid alone over 60 minutes
At each time, a greater degradation of hyaluronic acid alone compared to hyaluronic acid in the presence of excipient the being the case whatever the excipient studied.
All the excipients used protect the hyaluronic acid from degradation linked to oxidative stress, the greatest protective effect being observed in the presence of glycerol 100 and CMC.

1. A composition comprising at least hyaluronic acid and at least one polyol and/or carboxymethyl cellulose (CMC).

2. The composition according to claim 1, characterized in that the hyaluronic acid has a molecular weight between 10⁵ and 10⁷ Da.

3. The composition according to claim 1, characterized in that the polyol is a saturated or unsaturated, linear, branched or cyclic alkyl compound with at least two —OH functions on the alkyl chain, as well as polymers (polyethers) of these polyhydroxylated alkyl compounds.

4. The composition according to claim 3, characterized in that the polyol is an alkyl compound having from 2 to 12 carbon atoms.

5. The composition according to claim 1, characterized in that the polyol is selected from ethylene glycol [(HOCH₂—CH₂OH)], diethylene glycol [(HOCH₂—CH₂—O—CH₂—CH₂OH)], triethylene glycol [(HOCH₂—CH₂—O—CH₂—CH₂OCH₂—CH₂OH], propylene glycol [(propane-1,2-diol: HOCH₂—CHOH—CH₃)], trimethylene glycol [(propane-1,3-diol: HOCH₂—CH₂—CH₂OH)]; propylene glycol, polymers and copolymers of glycerol, ethylene glycol and propylene glycol, dipropylene glycol, and hexaglycerol, hexylene glycol, pentylene glycol, butyldiglycol, 1,2,3trihydroxyhexane, butylene glycol [(butane-1,3-diol], n-butylene glycol [(butane-1,4-diol], 2,3-butylene glycol [or secbutylene glycol (butane-2,3-diol)], Triols, glycerol; Tetraols, Erythritol, Threitol, Pentols (pentanols), Xylitol, Arabitol (lyxitol), Ribitol (adonitol); Hexols, Sorbitol (Gulitol), Dulcitol (Galactitol), Mannitol, Fucitol, Iditol; Heptols, Volemitol; C₁₂ Isomalt, C₁₂ Maltitol, C₁₂ Isomaltitol, C₁₂ Lactitol (lactositol), C₁₈ Maltotriitol, C₂₄ Maltotetraitol; and Polyglycitol.

6. The composition according to claim 1, characterized in that the hyaluronic acid is present in the composition in an amount comprised between 0.01% and 20% of the total weight of the composition.

7. The composition according to claim 1, characterized in that the polyol is present in the composition in an amount between 0.05 and 90% of the total weight of the composition.

8. The composition according to claim 1, characterized in that the carboxymethyl cellulose is present in the composition in an amount between 0.1% and 72% of the total weight of the composition.

9. The composition according to claim 1, characterized in that the ratio between the hyaluronic acid and the polyol in the composition is between 0.0001 and 400.

10. The composition according to claim 1, characterized in that the ratio between the hyaluronic acid and the carboxymethyl cellulose in the composition is between 0.0001 and 200.

11. The composition according to claim 1, characterized in that the ratio in the composition between the polyol and the carboxymethyl cellulose is between 0.0007 and 900.

12. A method comprising the use of at least one polyol and/or carboxymethyl cellulose to slow down, limit or even eliminate the degradation of hyaluronic acid, in a composition.

13. The method according to claim 12, to slow down, limit or even eliminate the degradation of the hyaluronic acid induced by ionizing radiation.

14. The method according to claim 12, to slow down, limit or even eliminate the degradation of the hyaluronic acid induced by oxidative stress.

15. The composition according to claim 1, comprising hyaluronic acid and at least one polyol and carboxymethyl cellulose.

16. The composition according to claim 2, characterized in that the hyaluronic acid has a molecular weight between 5.10⁵ and 2.10⁶ Da.

17. The composition according to claim 4, characterized in that the polyol is an alkyl compound having 2 or 3 carbon atoms.

18. The composition according to claim 6, characterized in that the hyaluronic acid is present in the composition in an amount comprised between 0.05% and 1% of the total weight of the composition.

19. The composition according to claim 8, characterized in that the carboxymethyl cellulose is present in the composition in an amount between 1 and 5% of the total weight of the composition.

20. The composition according to claim 10, characterized in that the ratio between the hyaluronic acid and the carboxymethyl cellulose in the composition is between 0.01 and 1.
